(19) United States
(12) Patent Application Publication
(10) Pub. No.: US 2011/0014305 A1
(43) Pub. Date: Jan. 20, 2011

(54) Extracts of eleutherococcus spp., preparation method thereof and use of the same

(75) Inventors: Chi-Hua Chen, Hsinchu City (TW); Shiow-Wen Chen, Hsinchu City (TW); Yuarn-Yee Chang, Hsinchu City (TW); Hui-Yun Tsai, Hsinchu City (TW)

Correspondence Address: Olson & Cepuritis, LTD., 20 NORTH WACKER DRIVE, 36TH FLOOR, CHICAGO, IL, 60606, US

(73) Assignee: Food Industry Research and Development Institute, Hsinchu City (TW)

(21) Appl. No.: 12/460,194

(22) Filed: Jul. 15, 2009

(51) Int. Cl. A61K 36/254

(52) U.S. Cl. 424/725

(57) ABSTRACT

The present invention relates to extracts of Eleutherococcus spp. and the preparation process thereof. The present invention also relates to the use of the Eleutherococcus spp. extracts of the present invention for the treatment or prevention of at least one condition of metabolic syndrome.

### FIELD OF THE INVENTION

The present invention relates to a herb extract and the preparation method thereof. The present invention also relates to the use of the extract for the treatment or prevention of metabolic syndrome.

### BACKGROUND OF THE INVENTION

Metabolic syndrome, also called “syndrome X,” “insulin resistance syndrome,” “Reaven's syndrome,” and “deadly quartet,” represents a constellation of risk factors for cardiovascular diseases. Several definitions of metabolic syndrome have been proposed by, among others, the National Cholesterol Education Program Adult Treatment Panel III (NCEP), the World Health Organization (WHO), International Diabetes Federation (IDF), and the American College Endocrinology. Generally, metabolic syndrome can be defined as having at least 2 or more of the characteristics including central obesity (waist circumference), elevated blood pressure (e.g., ≧140/90 mmHg), triglycerides, and fasting plasma glucose and decreased levels of high-density lipoprotein (HDL) cholesterol. Individuals who have the metabolic syndrome are at increased risk for type 2 diabetes as well as cardiovascular disease.

Generally, the individual disorders of the metabolic syndrome are treated separately by different drugs. Diruetics and ACE inhibitors may be used to treat hypertension. Cholesterol drugs may be used to lower LDL cholesterol and triglyceride levels if they are elevated, and to raise HDL levels if they are low. Drugs that ameliorate insulin resistance, for example, metformin and thiazolidinediones, may be utilized.

Eleutherococcus senticosus (Ruper. Et Maxim.) Maxim, originally named Acanthopanax senticosus (Araliaceae), is widely used in Chinese medicine alone or in combination with other herbs, to treat diseases. JP2003-277282 and JP2006-234603 disclose methods for extracting pharmaceutical compounds from Acanthopanax senticosus Harms. JP2007-277128 relates to the utilization of the powder of Eleutherococcus senticosus as one of the active ingredients of a Chinese medicine for treating obesity, diabetes and hyperlipidemia. JP2006-348054 suggests that the water, alcohol, ether, and acetone extracts of Acanthopanax dieboldianus have an effect in inhibiting lipase and that the extracts are effective for preventing or treating obesity and hyperlipemia. However, the specification of JP2006-348054 can only prove that the water extract had an IC₅₀ value of 170 μg/0.5 ml in inhibiting activity of lipase in vitro. Hikino H. et al. (J. Nat. Prod., 1986, 49(2):293-7) and Medon P. J. et al. (Zhongguo Yao Li Xue Bao, 1981, 2(4):281-5) disclose that the administration of Eleutherococcus senticosus extract effectively diminished plasma glucose level in mice. CN 101356968 discloses an extract of Polygonum multiflorum Thunb. ex Murray var. hypoleucum (Ohwi) having an effect in the improvement of metabolic syndrome.

There are more and more patients with metabolic syndrome. The present invention addresses treatment of the syndrome.

### SUMMARY OF THE INVENTION

One of the purposes of the present invention is to provide a process for preparing Eleutherococcus spp. extracts.

Another purpose of the present invention is to provide an Eleutherococcus spp. extract.

Another purpose of the present invention is to provide a composition comprising the Eleutherococcus sp. extract of the present invention.

Another purpose of the present invention is to provide a method for the prevention or treatment of at least one condition of metabolic syndrome in a subject, comprising administrating the composition of the present invention to a subject in need thereof.

Another purpose of the present invention is to provide a method for the prevention or treatment of diseases associated with adiponectin in a subject, comprising administrating the composition of the present invention to a subject in need thereof.

Another purpose of the present invention is to provide a method for the prevention or treatment of diseases associated with peroxisome proliferator-associated receptor γ (PPARγ) in a subject, comprising administrating the composition of the present invention to a subject in need thereof.

Another purpose of the present invention is to provide a method for reducing oxidative stress in a subject, comprising administrating the composition of the present invention to a subject in need thereof.

A further purpose of the present invention is to provide a use of the Eleutherococcus sp. extract of the present invention in the manufacture of a medicament for the prevention or treatment of at least one condition of metabolic syndrome.

A further purpose of the present invention is to provide a use of the Eleutherococcus sp. extract of the present invention in the manufacture of a medicament for the prevention or treatment of diseases associated with adiponectin.

A further purpose of the present invention is to provide a use of the Eleutherococcus sp. extract of the present invention in the manufacture of a medicament for the prevention or treatment of diseases associated with PPARγ.

A further purpose of the present invention is to provide a use of the Eleutherococcus sp. extract of the present invention in the manufacture of a medicament for reducing oxidative stress.

The present invention is described in detail in the following sections. Other characteristics, purposes and advantages of the present invention can be easily found in the detailed descriptions and claims of the invention.

### BRIEF DESCRIPTION OF THE DRAWINGS

FIG. 1 shows that the extract of E. giraldii not only activate PPARγ activity, but also increases the production of PPARγ in 3T3-L1 cells. “Trog” represents troglitazone and “EA16” represents the 50% EtOH-EtOAc extract of E. giraldii.

FIG. 2 shows results of oral glucose tolerance test in SD rats at week 4. The values are shown as mean±SD (n=7-8). *p<0.05 compared with group C. #p<0.05 compared with group F.

### DETAILED DESCRIPTION OF THE INVENTION

Unless otherwise defined herein, scientific and technical terms used in connection with the present invention have the meanings that are commonly understood by those of ordinary skill in the art. The meaning and scope of the terms should be clear; however, in the event of any latent ambiguity, definitions provided herein take precedence over any dictionary or extrinsic definition.

Unless otherwise required by the context, singular terms include the plural and plural terms include the singular.

As utilized in accordance with the present disclosure, the following terms, unless otherwise indicated, shall be understood to have the following meanings.

The term “preventing” or “prevention” as used herein refers to delaying the onset of the symptoms in a susceptible subject or reducing the occurrence of a disease.

The term “treating” or “treatment” as used herein denotes reducing and/or improving the symptoms in a susceptible subject.

The term “subject” as used herein denotes animals, especially mammals. In one preferred embodiment, “subject” denotes “humans.”

The term “therapeutically effective amount” as used herein refers to the amount of an active ingredient used alone or in combination with other treatments/medicaments for treating the same disease that shows therapeutic efficacy.

The term “carrier” or “pharmaceutically acceptable carrier” refers to diluents, excipients, acceptors or analogues used for manufacturing pharmaceutical compositions which are well known to persons of ordinary skill in the art.

The term “Eleutherococcus sp.” refers to the roots, leaves, stems, and/or the whole plant of Eleutherococcus spp., preferably, E. senticosus (Ruper. et Maxim.) Maxim., E. giraldii, or E. trifoliatu. According to the invention, the plant sample can be unprocessed or processed, e.g., dried, sliced, ground, etc.

### The Preparation Processes

The process for preparing an Eleutherococcus sp. extract of the subject application comprises the following steps:
a) extracting Eleutherococcus sp. with an alcohol solution; and
b) removing the solid portion from the extract obtained from step a) and thereby obtaining the Eleutherococcus sp. extract.

According to the process of the present invention, the ratio of the weight of Eleutherococcus sp. to the volume of the alcohol solution is about 1/5 to about 1/50, preferably about 1/10 to about 1/30, and most preferably about 1/20.

According to the process of the present invention, the term “alcohol solution” refers to an absolute alcohol or a solution prepared by mixing alcohol with water. According to the invention, the alcohol has a straight or branched alkyl portion having one to six carbon atoms, preferably having one to four carbon atoms, and more preferably having one to three carbon atoms. For example, the alcohol is methanol, ethanol, isopropanol or a mixture thereof. Preferably, the alcohol is methanol or ethanol.

The concentration of alcohol in the alcohol solution used in the process of the present invention is about 30 to 100 v/v %. In one preferred embodiment, the alcohol solution is a methanol solution of about 30 to 100 v/v %, preferably about 40 to 90 v/v %, and most preferably about 50 to 70 v/v %. In another preferred embodiment, the alcohol solution is an ethanol solution of about 30 to 100 v/v %, preferably about 40 to 90 v/v %, and most preferably about 50 to 70 v/v %.

According to the process of the present invention, the extraction of step a) can be conducted with any conventional methods, such as decoction, dipping, sonication, stirring, agitation, or the combination thereof. The extraction period is about 1 to about 48 hours, preferably from about 12 to 36 hours, and most preferably about 24 hours. The extraction temperature is from about 20 to 45° C., and preferably about 25 to 35° C.

According to the process of the present invention, in step b), the solid portion can be removed with any conventional methods, such as filtration through cheesecloth, centrifugation, or the combination thereof.

The process of the present invention may further comprise c) concentrating the Eleutherococcus sp. extract with any conventional methods, such as freeze-dry or evaporation (J. Pharmacol. Sci., 99:294-300, 2005; and J. Chromatography, 932:91-95, 2001), to obtain a concentrated Eleutherococcus sp. extract.

According to the process of the present invention, the concentrated Eleutherococcus sp. extract from step c) can be further extracted/partitioned with a solvent having medium-to-high polarity to obtain a second Eleutherococcus sp. extract. According to the invention, the solvent having medium-to-high polarity can be any known in the art, such as an ether, an alcohol, an ester or a mixture thereof. Examples of the solvent include, but are not limited to, isobutyl alcohol, n-butanol, n-butyl acetate, chloroform, ethyl acetate, and mixtures thereof. The v/v ratio of the concentrated Eleutherococcus sp. extract to the solvent is about 1:1 to 1:4.

In a preferred embodiment of the process of the present invention, the solvent is ethyl acetate, and the extraction is conducted by liquid-liquid extraction (Free Radic Res., 38(1):97-103, 2004; and J. Herb. Pharmacother., 7:107-128, 2007). Generally, the concentrated Eleutherococcus sp. extract is contacted with ethyl acetate, and then 0.5% sodium biocarbonate is added to the ester layer at v/v ratio of about 1:1 to remove fatty acids (Biosci. Biotechnol. Biochem. 62(3):532-534, 1998).

### Compositions

The present invention provides a composition comprising a therapeutically effective amount of the Eleutherococcus sp. extract of the present invention. The composition of the present invention can be used as a food composition or a pharmaceutical composition.

The composition of the present invention can be administrated to a subject by any suitable administration route, such as oral administration. Suitable formulations include but are not limited to tablets, lozenges, hard or soft capsules, aqueous or oily suspensions, emulsions, dispersible powders or granules, syrups or elixirs. If necessary, it may be sterilized or mixed with any pharmaceutically acceptable carriers, such as stabilizers, wetting agents and the like.

The compositions of the invention may be obtained through conventional procedures using conventional pharmaceutical excipients that are well known in the art. Thus, compositions intended for oral use may contain, for example, one or more coloring, sweetening, flavoring and/or preservative agents.

The composition of the present invention may be used in combination with one or more other current medicaments for treating metabolic syndrome, such as lipid-lowering drugs, antidiabetics agent, and antioxidant agent (Cardiovasc Hematol Agents Med Chem. 2008,6(4):237-52).

### Utilities

The compositions of the present invention can be used for the prevention or treatment of at least one, preferably two or more conditions of metabolic syndrome. The core conditions of the metabolic syndrome include, but are not limited to, obesity, central adiposity (abdominal obesity), dyslipidemia, impaired glucose tolerance, cardiovascular disease, insulin resistance, and type 2 diabetes. (Life Sci. 2003, 73:2395-2411)

The compositions of the present invention can be used for the inhibition of the activity of acetyl-CoA carboxylase. Acetyl-CoA carboxylase is one of the key enzymes involved in fatty acid synthesis. The product of acetyl-CoA carboxylase reaction, malonyl-CoA, is both a substrate of fatty acid synthase (FAS) and an inhibitor of camitin palmitoyl CoA transferase (CPT). Therefore, the inhibition of ACC not only reduces the synthesis of triglycerides, but also lowers the synthesis of fatty acid. (Kusunoki et al., Endocrine, 29:91-100, 2006; Abu-Elheigh et al., PNAS, 100:10207-10212, 2003; Arbeeny et al., J. Lipid Res., 33(6): 843-851, 1992; Rose-Kahn and Bar-Tana., Biochim. Biophys. Acta. 1042:259-264, 1990; and Harwood, Expert. Opin. Ther. Targets, 9(2):267-281, 2005)

The compositions of the present invention can be used for the prevention or treatment of diseases associated with adiponectin. The diseases associated with adiponectin include, but are not limited to, obesity, insulin resistance, hyperglycemia, and atherosclerosis. (J. Cardiometab. Syndr., 2007, 2(4):288-94; and J. Clin. Endocrinol. Metab., 2005, 90:4792-4796)

The compositions of the present invention can be used to activate PPARγ. The activation of PPARγ can induce the differentiation of preadipocyte and the metabolism of fats, and improve the insulin sensitivity of cell. (Endocrine Rev., 1999, 20:649-688)

The composition of the present invention can be used to scavenge reactive oxygen species (ROS), like antioxidants. The ROS overproduction is detrimental and toxic to cells and tissues, which plays an important role in the development of diabetes, atheroscieropathy, nephropathy, neuropathy, and retinopathy (Am. J. Nephrol., 29:62-70, 2009; and Cardiovascular Diabetology, 1:3-32, 2002). Dietary antioxidants, which have a protective role against oxidative stress, have been proposed as therapeutic agents to counteract liver damage (Crit. Rev. Food Sci. Nutr., 44:575-586, 2004), retinal damage (Diabetes Metab. Rev., 22:38-45, 2006), and atherosclerosis (J. Pharmacol. Sci., 99:294:-300, 2005).

Persons skilled in the art should have no difficulty choosing the suitable routes and dosages for treatments. According to the present invention, the preferred route is oral administration. Dosage will depend on the nature and condition of the disorder, age and health condition of the patient, administration route and any previous treatment. Persons skilled in the art should know that dosage may vary depending on the individual's age, size, health condition and other related factors.

The following examples are provided to aid those skilled in the art in practicing the present invention. The examples should not be construed as unduly limiting the present invention, as modifications to and variations on the embodiments discussed herein may be made by those having ordinary skill in the art without departing from the spirit or scope of the present discovery.

### EXAMPLES

### Example 1

### Preparation of Extracts of Eleutherococcus Spp.

### i) Alcohol Extracts

Stems of each Eleutherococcus spp., including Eleutherococcus senticosus (Ruper. et Maxim.) Maxim., E. giraldii, and E. trifoliatus, were machine grounded, and then extracted with a solvent, e.g., 50% or 70% of methanol or ethanol, at a weight to volume ratio of about 1/20 for 24 hour, filtered through 4 layers of cheesecloth, and concentrated by a pressure reduce condenser.

### ii) Ethyl Acetate (EtOAc) Extract

The concentrated MeOH— and EtOH-extracts prepared as above were extracted with an equal volume of ethyl acetate. The ethyl acetate layers were treated with 5% of sodium bicarbonate to remove fatty acids, and then were concentrated to obtain the MeOH— or EtOH-EtOAc extracts.

### Example 2

### Inhibitory Effects of the Extracts of Eleutherococcus Spp. on Acetyl-CoA Carboxylase (ACC)

ACC was isolated and purified from rat liver, and the inhibition on ACC activity by the extracts obtained from Example 1 was assayed as previously described by Tanabe et al. (1981. Methods in Enzymology, 71(Pt C):5-16.) As shown in Table 1, all the extracts showed inhibition effect on ACC activity.

TABLE 1
Inhibitory effects of extracts of Eleutherococcus spp. on ACC activities.

Inhibition of ACC (%)

70% Ethanol Extracts (at 0.5 mg/mL)

| | |
|---|---|
| Eleutherococcus senticosus | 38 |
| (Ruper. et Maxim.) Maxim. | |
| E. giraldii | 25 |
| E. trifoliatus | 19 |

Ethyl Acetate Extracts of E. giraldii (at 0.05 mg/mL)

| | |
|---|---|
| 50% MeOH-EtOAc | 16 |
| 50% EtOH-EtOAc | 28 |
| 70% EtOH-EtOAc | 30 |

### Example 3

### Effects of E. giraldii Extracts on the Differentiation of 3T3-L1 Preadipocytes

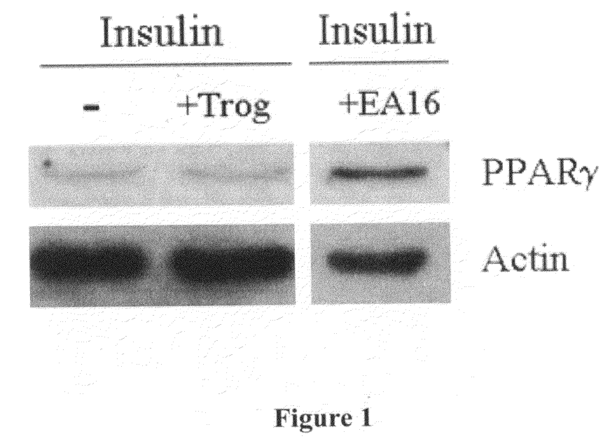

As described in Waki et al. (2007. Cell Metab. 5:357-370), 3T3-L1 cells were cultured in DMEM (4,500 mg/L glucose) supplemented with 10% fetal bovine serum, 2 mM glutamine, 100 units/ml penicillin, 100 μg/ml streptomycin, and 110 μg/ml sodium pyruvate, and cultured in a 5% CO₂ incubator at 37° C. The differentiation of 3T3-L1 cells into adipocytes was initiated by the addition of 10 μg/mL insulin (day 0) to the cultural medium. After incubation in the insulin containing medium for 7 days (at day 2 and day 4, the medium was refreshed), the cells were incubated in a medium without insulin for an additional 2 days. Extracts of E. giraldii were also added to the medium on day 0 and when the medium was refreshed. The amounts of triglyceride (TG) and adiponectin in the test cells and the binding activity of PPARγ of the cells were assayed with commercial assay kits (Quantikine Mouse Adiponectin immunoassay from R&D Systems, Triglycerol assay Kit from Audit Diagnostics, Ltd., and BD™ Transfactor Family Colorimetric Kits—PPARαβγ (BD Biosciences) or Transcription Factor PPARγ ELISA Kit (Panomics), respectively), and the expressions of PPARγ were determined by Western blot. As shown in Table 2, the extracts of E. giraldii could not only dose-dependently stimulate the differentiation of 3T3-L1 preadipocytes, but also stimulate the production of both the secreted adiponectin and the intracellular adiponectin. FIG. 1 shows that, unlike the known PPARγ agonist, troglitazone, the extract of E. giraldii could not only activate the PPARγ activity, but also increase the production of PPARγ in 3T3-L1 cells.

TABLE 2
Effects of E. giraldii extracts on the differentiation of 3T3-L1
preadipocytes.

| | 70% Ethanol Extract of | | | |
|---|---|---|---|---|
| | E. giraldii (μg/mL) | | | |
| | 0 | 20 | 40 | 100 |
| TG (μg/mg of protein) | 70.1 | 78.7 | 91.6 | 96.3 |
| Secreted adiponectin (ng/mL) | 104 | 125 | 135 | 153 |
| Intracellular adiponectin (ng/mL) | 2.8 | 2.7 | 4.0 | 4.1 |
| | 0 | 10 | 20 | 40 |
| | 50% EtOH-EtOAc Extract of | | | |
| | E. giraldii (μg/mL) | | | |
| TG (μg/mg of protein) | 127 | 175 | 178 | 241 |
| Secreted adiponectin (ng/mL) | 82 | 213 | 392 | 574 |
| Intracellular adiponectin (ng/mL) | 2.8 | 6.8 | 8.1 | 6.8 |
| Absorbance at 450 nm for binding | 0.495 | 0.462 | 0.604 | 0.967 |
| activity to PPARγ (ELISA assay) | | | | |
| | 70% EtOH-EtOAc Extract of | | | |
| | E. giraldii (μg/mL) | | | |
| TG (μg/mg of protein) | 127 | 154 | 219 | 301 |
| Secreted adiponectin (ng/mL) | 90 | 336 | 425 | 634 |
| Intracellular adiponectin (ng/mL) | 2.8 | 6.8 | 7.2 | 5.3 |

### Example 4

### Effects of 70% Ethanol E. giraldii Extract on Male Sprague-Dawley (SD) Rats Having Metabolic Syndrome Induced by High-Fructose Diet

Animal studies were performed to assess the effects of E. giraldii extracts in the prevention and treatment of metabolic syndrome. Two hundred gram-, male SD rats (purchased from BioLASCO Taiwan Co., Ltd.) were kept 2 per microisolator cage in a temperature-controlled room at 23±2° C. with a fixed 12 h/12 h light/darkness cycle. The animals were allowed free access to water and food.

The rats were divided into four groups (n=8 in each group). Normal control (C) group was fed with regular animal chow diet (Altromin 1362N, Altromin, Im Seelenkamp, Germany). The other three groups were given a 40% high-fructose diet so as to induce metabolic syndrome, while L and H groups was infused daily with 94 and 188 mg/kg of 70% ethanol E. giraldii extract, respectively, and high-fructose control (F) group was infused with the same volume of distilled water.

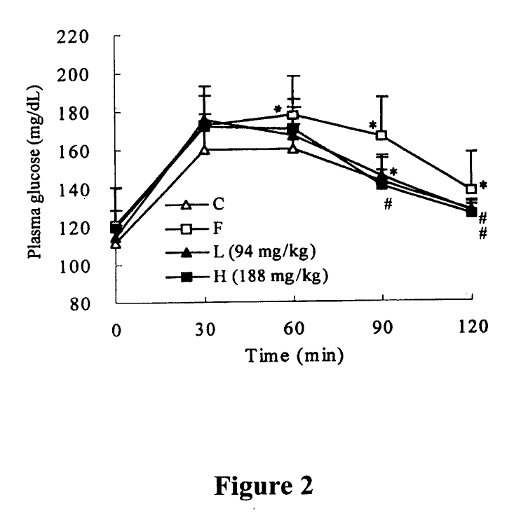

Body weight and food consumption were measured. Body weight was measured at 9:00-11:00 AM. Oral glucose tolerance tests (OGTTs) were performed on weeks 4 and 8. Rats were starved overnight for 16 hours before the test. At the beginning of the tests (time 0), the rats were weighed and 0.5 mL of blood sample was collected from tail vein, and then the rats received an oral infusion of 10% of D-glucose (1.5 g/kg body weight). At 30, 60, 90, and 120 minutes after infusion, further blood samples (0.5 mL) were collected. The results of OGTT performed on week 4 are shown in FIG. 2. It can be seen from FIG. 2 that at 60, 90, and 120 minutes, the concentrations of plasma glucose of group F were higher than those of group C (p<0.05), and that at 90 and 120 minutes, the concentrations of plasma glucose of groups L and H were significantly lower that those of group F (p<0.05). The results in week 8 are similar to those in week 4.

10 weeks later, the rats were sacrificed, and then epididymal fat pads, livers and kidneys were dissected out and weighed. Table 3 shows that after 10 weeks of dietary treatments, the relative weights of epididymal fat pads of groups L and H were all significantly lower than that of group F. No abnormalities in organ weight were observed in the necropsy performed at the end of the experiment. The concentrations of liver triglyceride were also measured, and it was found that the concentrations of H group were significantly lower than those of F group (10.7±1.7 mg/g liver vs. 12.5±1.4 mg/g liver, p<0.05) after 10 weeks of dietary manipulation.

As a result, it is demonstrated that the E. giraldii extract exhibits dramatic effects on improving glucose tolerance in vivo and reducing the weight of epididymal fat.

TABLE 3
Relative tissue weights of SD rats after 10 weeks of dietary treatments.

| | Relative Tissue Weight | | |
|---|---|---|---|
| | (g/100 g body weight) | | |
| Group | Epididymal Fat Pads | Liver | Kidney |
| C | 1.17 ± 0.17 | 2.67 ± 0.22 | 0.67 ± 0.07 |
| F | 2.39 ± 0.29* | 2.87 ± 0.33 | 0.71 ± 0.05 |
| L (94 mg/kg) | 1.97 ± 0.27*^{#} | 3.03 ± 0.43 | 0.73 ± 0.04 |
| H (188 mg/kg) | 1.87 ± 0.33*^{#} | 2.85 ± 0.23 | 0.69 ± 0.05 |

Values are shown as mean ± SD (n = 7~8).
*p < 0.05 compared with group C.
^{#}p < 0.05 compared with group F.

### Example 5

### Effect of Extracts of Eleutherococcus Spp. in the Reduction of High Glucose-Induced Triglyceride Accumulation in HepG2 Cells

HepG2 cells were seeded (2.5×10⁵ cells/well) in DMEM-LG (containing 5.5 mM glucose) and incubated in a chamber with a temperature of 37° C., a humidity of 95% and an atmosphere of 5% CO₂ for 24 hours. The media in the wells were replaced with either fresh DMEM-LG, DMEM-HG (containing 50 mM glucose), or DMEM-HG-EXTRACT (containing 50 mM glucose and different extracts of Eleutherococcus spp.) and the cells were further incubated for 120 hours. The cells were washed twice with D-PBS, and treated with lysis buffer (50 mM Hepes, 1% Triton X-100, 150 mM NaCl, 2 mM NaVO₄, and 1 mM PMSF). The concentrations of triglyceride (TG) and protein were measured with commercial kits (Triglyceride GPO-PAP kit from E. Merck, Darmstadt, Germany, and Bio-Rad Protein assay kit from Bio-Rad, Hercules, Calif., USA, respectively). As shown in Table 4, the 70% ethanol extracts of E. trifoliatus and E. giraldii decreased the accumulation of cellular TG of HepG2 incubated in high glucose; and the extent of inhibition was further enhanced by ethyl acetate extraction.

TABLE 4
Inhibition on the high glucose-induced triglyceride accumulation in
HepG2 cells by the extracts of Eleutherococcus spp.

| | TG (mg/mg of protein) | Inhibition %* |
|---|---|---|
| Low glucose (5 mM) | 91.58 | |
| High glucose (50 mM) | 117.57 | |

70% EtOH EXTRACTS

| | | |
|---|---|---|
| E. trifoliatus (10 μg/ml) | 114.44 | 12.0% |
| E. giraldii (10 μg/ml) | 109.45 | 31.2% |
| E. giraldii (20 μg/ml) | 103.87 | 52.7% |

50% EtOH-EtOAc EXTRACT

| | | |
|---|---|---|
| E. giraldii (10 μg/ml) | 101.65 | 61.3% |

*Inhibition % = (TG_{extract} − TG_{high glucose})/(TG_{low glucose} − TG_{high glucose}) × 100

### Example 6

### Antioxidative Activities of the Extracts of Eleutherococcus Spp.

In vitro total antioxidant activity was measured using oxygen-radical absorbance capacity (ORAC) method (Ou et al., 2001, J. Agric. Food Chem., 49: 4619-4626; and Huang et al., 2002, J. Agric. Food Chem., 50:4437-4444). After 20 μl of blank (75 mM phosphate buffer), 0˜200 μM trolox (6-Hydroxy-2,5,7,8-tetramethylchroman-2-carboxylic acid (Aldrich, Wis., USA) or test samples with different extracts of Eleutherococcus spp. were added to different wells of a 96-well black plate, 150 μl of 96 nM disodium fluorescein (Aldrich) were added to the wells. The plate was placed in Infinite M200 fluorescence microplate reader (TECAN) for mixing. Thirty μl of 320 mM AAPH (2,2′-azobis (2-amidinopropane)dihdrochloride, Aldrich) were added, and the plate was returned for reading at excitation 490 nm, emission 530 nm. All ethanol extracts of Eleutherococcus spp. showed relative antioxidant activities (Table 5). With the exception of the E. trifoliatus extracts, the antioxidative activities of ethanol extracts were greater than those of their respective ethyl acetate extract counterparts.

TABLE 5
Antioxidative activities of extracts of Eleutherococcus spp.

ORAC (μmole TE/g ext.)*

| | | 70% EtOH- |
|---|---|---|
| | 70% EtOH extract | EtOAc extract |
| E. giraldii | 2930 ± 70 | 1070 ± 1000 |
| E. senticosus | 3010 ± 10 | 1230 ± 190 |
| E. trifoliatus | 2700 ± 1040 | 2650 ± 720 |

*TE—trolox equivalents
Results are given as mean ± SD of three experiments.

A further antioxidant activity of the 50% EtOH-EtOAc E. giraldii extract was assayed as described by Puhl et al. (1994. Methods in Enzymology 233: 425-41). Human LDL (d=1.019-1.063 g/mL) was isolated by sequential ultracentrifugation from the plasma samples of consenting normolipidemic human subjects after overnight fasting (Miyazaki et al., 1994. J Biol. Chem. 269: 5264-9). LDL was dialyzed against PBS containing 0.15 mol/L NaCl and 1 mmol/L EDTA (pH 7.4). One hundred and fifty μl of the dialyzed LDL sample (100 μg of protein/mL) were pre-incubated with each of the various concentrations of extract, and then 5 μM CuSO₄ (final concentration) was added. The amounts of the conjugated diene formed were monitored at 232 nm. It was found that that the lag time was prolonged from 58 min (blank) to 268 min in the presence of 50% EtOH-EtOAc extract of E. giraldii (at 20 g/ml).

What is claimed is:

1. A process for preparing an Eleutherococcus spp. extract comprising the steps of:
a) extracting Eleutherococcus spp. with an alcohol solution; and
b) removing the solid portion from the extract obtained from step a) and thereby obtaining the Eleutherococcus spp. extract.

2. The process of claim 1, wherein Eleutherococcus spp. is Eleutherococcus senticosus (Ruper. et Maxim.) Maxim., E. giraldii, or E. trifoliatus.

3. The process according to claim 1, wherein the ratio of the weight of Eleutherococcus spp. to the alcohol solution is about 1/5 to about 1/50.

4. The process according to claim 1, wherein the alcohol solution is an ethanol, methanol or isopropanol solution.

5. The process according to claim 1, wherein the concentration of the alcohol is about 30 to 100 v/v %.

6. The process according to claim 1, which further comprises c) concentrating the Eleutherococcus spp. extract.

7. The process according to claim 6, which further comprises d) extracting/partitioning the concentrated Eleutherococcus spp. extract with a solvent having medium-to-high polarity to obtain a second Eleutherococcus spp. extract.

8. The process according to claim 7, wherein the solvent having medium-to-high polarity is selected from isobutyl alcohol, n-butanol, n-butyl acetate, chloroform, ethyl acetate or a mixture thereof.

9. The process according to claim 8, wherein the solvent having medium-to-high polarity is ethyl acetate.

10. An Eleutherococcus spp. extract prepared by the process according to claim 1.

11. An Eleutherococcus spp. extract prepared by the process according to claim 6.

12. An Eleutherococcus spp. extract prepared by the process according to claim 7.

13. A composition comprising a therapeutically effective amount of the Eleutherococcus spp. extract according to claim 10 and optionally a pharmaceutically acceptable carrier, diluent or excipient.

14. A composition comprising a therapeutically effective amount of the Eleutherococcus spp. extract according to claim 11 and optionally a pharmaceutically acceptable carrier, diluent or excipient.

15. A composition comprising a therapeutically effective amount of the Eleutherococcus spp. extract according to claim 12 and optionally a pharmaceutically acceptable carrier, diluent or excipient.

16. A method for preventing or treating at least one condition of metabolic syndrome in a subject in need thereof comprising administering a composition according to claim 13 to the subject.

17. A method for preventing or treating at least one condition of metabolic syndrome in a subject in need thereof comprising administering a composition according to claim 14 to the subject.

18. A method for preventing or treating at least one condition of metabolic syndrome in a subject in need thereof comprising administering a composition according to claim 15 to the subject.

19. The method according to claim 16, wherein the condition is central adiposity (abdominal obesity), lipid abnormalities, cardiovascular disease, impaired glucose tolerance, or insulin resistance.

20. The method according to claim 17, wherein the condition is central adiposity (abdominal obesity), lipid abnormalities, cardiovascular disease, impaired glucose tolerance, or insulin resistance.

21. The method according to claim 18, wherein the condition is central adiposity (abdominal obesity), lipid abnormalities, cardiovascular disease, impaired glucose tolerance, or insulin resistance.

22. A method for inhibiting the activity of acetyl-CoA carboxylase in a subject in need thereof comprising administering a composition according to claim 13 to the subject.

23. A method for inhibiting the activity of acetyl-CoA carboxylase in a subject in need thereof comprising administering a composition according to claim 14 to the subject.

24. A method for inhibiting the activity of acetyl-CoA carboxylase in a subject in need thereof comprising administering a composition according to claim 15 to the subject.

25. A method for preventing or treating diseases associated with adiponectin in a subject in need thereof comprising administering a composition according to claim 13 to a subject.

26. A method for preventing or treating diseases associated with adiponectin in a subject in need thereof comprising administering a composition according to claim 14 to the subject.

27. A method for preventing or treating diseases associated with adiponectin in a subject in need thereof comprising administering a composition according to claim 15 to the subject.

28. A method for activating peroxisome proliferator-associated receptor γ in a subject in need thereof comprising administering a composition according to claim 13 to the subject.

29. A method for activating Peroxisome Proliferator-Associated Receptor γ in a subject in need thereof comprising administering a composition according to claim 14 to the subject.

30. A method for activating Peroxisome Proliferator-Associated Receptor γ in a subject in need thereof comprising administering a composition according to claim 15 to the subject.
